(19) United States
(12) Patent Application Publication
(10) Pub. No.: US 2022/0304592 A1
(43) Pub. Date: Sep. 29, 2022

(54) APPARATUS AND METHOD FOR INDUCTIVELY MEASURING A REPRESENTATIVE VARIABLE OR A VARIATION IN THE CIRCUMFERENCE OF A DEFORMABLE OBJECT, AND USE OF THE APPARATUS ON A SHUTTER, A PRESSURE PROBE OR FOR INDUCTANCE PLETHYSMOGRAPHY

(71) Applicant: CALYF, MAISONS LAFFITTE (FR)

(72) Inventor: Francis COUR, MAISONS-LAFITTE (FR)

(21) Appl. No.: 17/639,029

(22) PCT Filed: Aug. 28, 2020
(86) PCT No.: PCT/EP2020/074064

§ 371 Date: Feb. 28, 2022

(30) Foreign Application Priority Data
Aug. 29, 2019 (FR) FR19 09502

(51) Int. Cl. A61B 5/107; G01B 7/28

(52) U.S. Cl. CPC A61B 5/1072; G01B 7/282

(57) ABSTRACT

The measurement apparatus (50) comprises at least one hybrid elastic cable (52) wound around the deformable object (22), and a measurement unit (54) capable of measuring the inductance of a wire of the hybrid elastic cable (52) comprising a conductive material between two reference points (58).

In general, this invention concerns an inductive measurement apparatus, suited to measure the circumference, or changes in the circumference, of a deformable object. It also concerns the application of this apparatus for measuring the circumference of an inflatable object of the type used in pressiometric or dilatometric probes in the field of geotechnology, for which the expansion rates may range from a few percent to a few hundred percent, or an inflatable element such as an inflatable stopper or packer of the type used in the oil and gas, geothermal, or geotechnological fields.

More specifically, in a first aspect, the invention concerns an apparatus for measuring a variable representative of a parameter or a variation of a circumference of a deformable object, wherein the apparatus comprises an inductance measurement unit.

Pressiometric and dilatometric tests are load tests carried out in the subsoil, generally in a calibrated borehole, the analysis of which delivers the mechanical properties of the subsoil, e.g. the pressuremeter modulus EM. In the case of a dilatometer, the expansion measurement is carried out by displacement sensors; in the case of the pressuremeter, the expansion is deduced from the volume of the fluid injected in order to fill the probe.

In order to carry out measurements of this type, an object that is deformable by dilation (here, a deformable cell), is pressurised with increasing pressure increments by injecting the fluid or fluid reservoir by means of the transfer element of the transfer unit. For each pressure increment, the volume of fluid injected is measured.

In the case of a dilatometer, the expansion measurement is carried out by displacement sensors; in the case of the pressuremeter, the expansion is deduced from the volume of the fluid injected in order to fill the probe.

However, measurement devices of this type do not allow for precise measurements. Measurements carried out using a dilatometer are only precise at the points of contact of the sensors, of which there are generally three, and with deformations and deformations limited to expansion rates of a few tens of percents, at certain discrete points that do not allow for the characterisation of terrain, in particular when the terrain is heterogeneous (consisting of layers of non-homogeneous material).

One solution to this problem is to measure the circumference at different longitudinal heights of the deformable cell, which has a cross section with a circumference that varies depending on the type of subsoil material that is surrounding it, once the deformable cell is under pressure and as it is incrementally inflated. Thus, as a function of the circumference at different longitudinal heights of the deformable cell and of its pressure, it is possible to obtain precise subsoil measurements that take into account, inter alia, the vertical stratification of the subsoil.

It is possible to estimate the circumference of a cylinder by measuring the inductance of a conductive wire coiled around it by applying formulae for calculating the inductance of a short solenoid or a simple conductive loop. However, such circumference calculations are only applicable to fixed circumferences, and thus it is not feasible to use a conductor coiled around a probe positioned in the subsoil with a circumference that is capable of varying substantially on the order of 1:3 (i.e., expansion rate of 300%) because the conductors on the market do not have sufficient expansion rates.

When measuring a variable circumference, the principle of measuring the circumference via an inductance loop is used in the field of inductance plethysmography to estimate a circumference around a part of the body of an individual or animal or, more commonly, to estimate the corresponding cross-section. However, the sensors used in plethysmography are used for small deformations on the order of expansion rates in the tens of percent, and do not allow for measurements of bodies that may reach circumferential expansion rates of 1:3 (diameter expansion coefficient of 300%).

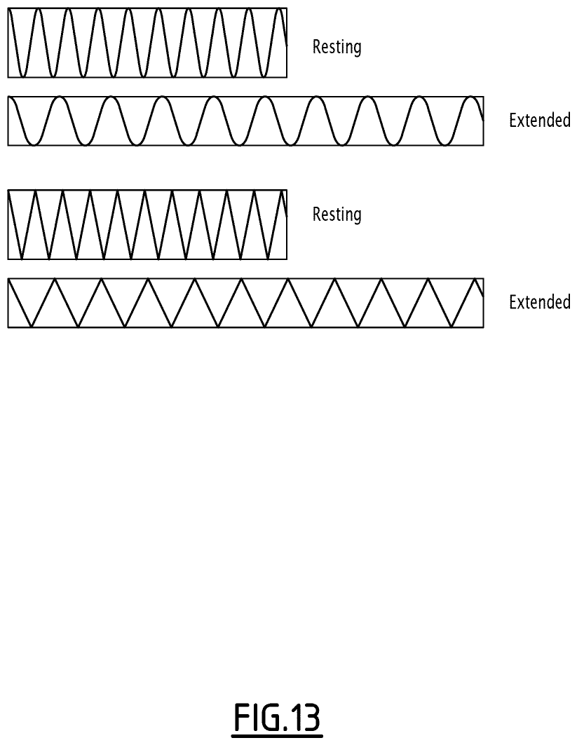

U.S. Pat. No. 5,913,830 describes an apparatus for measuring the circumference around a part of an individual's body by measuring the inductance of an electrical wire affixed to an elastic band, which is itself coiled around the part of the individual's body. However, such an apparatus does not allow for precise circumference measurements. Additionally, such devices do not allow for measurements over substantial expansion ranges, given that the precision of measurements with such devices decreases as the circumference being measured increases. Indeed, in the inductive bands used in inductance plethysmography (commonly known as ‘plethysmography belts’), an electrical wire is associated, e.g. sewn or bonded, onto an elastic band in a flat zigzag, sinusoidal, or other regular curved configuration over all or part of the length of the elastic band. By extending when stretched, the elastic band will take the conductive wire with it, thus eliminating and flattening the curves formed by the conductive wire (FIG. 13).

U.S. Pat. No. 4,308,872A discloses an inductance plethysmography apparatus using a helical electrical wire affixed at regular points to an elastic band.

The objective of this invention is to propose an apparatus for measuring a circumference or variation in a circumference of a deformable object that allows for precise measurement of a variable circumference without the need for adjustments or repositioning between different measurements for a wide range of circumferential expansion coefficients of up to several hundred percent.

To this end, in a first aspect, this invention concerns a measurement apparatus of the aforementioned type, characterised in that it further comprises at least one hybrid elastic cable that is coiled and arranged so as to form at least one turn around the deformable object, wherein the/each hybrid elastic cable comprises a wire of a first and a second type.
wherein, for the/each hybrid elastic cable, the wire of the first type has a degree of tenacity lower than that of the wire of the second type, wherein the wire of the second type has a degree of elasticity lower than that of the wire of the first type, wherein the wire of the second type comprises a conductive material,
wherein, for the/each hybrid elastic cable, the wire of the second type is coiled helically around the wire of the first type when the hybrid elastic cable is at rest,
wherein, for the/each hybrid elastic cable, the measurement unit comprises a pair of measuring terminals electrically connected to two reference points of the cable and is suited to measure the inductance of the wire of the second type of the cable between the two reference points of the cable,
wherein the apparatus further comprises a calculating unit electrically connected to the measurement unit and configured to calculate the representative variable for the/each cable around the cable using the inductance of the wire of the second type of the cable being measured.

Such an apparatus can measure the circumference or variations in the circumference of a deformable object with high precision over a wide range of expansion.

This is due, in particular, to the fact that the hybrid cable may undergo significant extension whilst maintaining the same general shape, and return to its initial position without residual deformation or elongation.

In particular, the wire of the second type, which includes the conductive material, remains arranged in a solenoid shape, with the solenoid maintaining substantially the same diameter.

The apparatus allows for measurements over a range of diameter expansions of up to a ratio of 1:3 without deterioration in the precision or quality of the measurements.

The measurement precision is less than a few hundred micrometres for the measurement of the circumference of a deformable object when this object has a circular cross section and a diameter on the order of 10-1000 mm, i.e. a precision of less than 0.1%.

Furthermore, the hybrid cable may undergo a large number of extension-retraction cycles, with each cycle having diameter expansion rates of several hundred percent, without being damaged, and in particular without any disturbance of the arrangement of the wire of the second time. Upon retraction, the wire of the first type, which is more elastic, returns the wire of the second type to its initial configuration in an ordered fashion.

Thus, the measurement apparatus allows for excellent measurement reproducibility without any degradation of precision.

The apparatus is extremely simple to use, in particular because it relies on existing technologies for inductance measurement using hybrid cable terminals, commonly known as ‘inductance meters’.

Furthermore, the hybrid cable has low extension resistance. High extension resistance could interfere with the measurement.

The inductive band of U.S. Pat. No. 4,308,872A does not make it possible to make precise and large amplitude measurements without degrading the geometry of the helical wire and therefore disturbing the repeated measurements. To date, this apparatus is not in use.

In the invention, the resting geometry of this hybrid wire does not deteriorate after cyclical stresses, and the hybrid wire does not need to be associated with an elastic band.

In particular embodiments, the measurement apparatus comprises one or more of the following characteristics, alone or in any combination technically possible:

the deformable object has a substantially circular cross section at the level of the/each hybrid elastic cable, and the calculating unit is configured to calculate the representative variable for a cable using a linear relationship between the inductance measured by the measuring unit and the representative variable;

when fully elongated, the hybrid cable is in a configuration in which the wire of the first type is coiled helically around the wire of the second type with a number of turns per linear metre of the hybrid cable between n_{sE}−15% and n_{sE}+15%, wherein n_{sE} is determined as a function of the diameter of the wire of the first type, the diameter of the wire of the second type, and a predetermined maximum elongation rate, based on the following formula:

$${n}_{sE} = \frac{1000}{π ( {φ}_{s} + {φ}_{K} )} \times \frac{\sqrt{{K}_{m ax} \times ( {K}_{m ax} + 200 )}}{{K}_{m ax} + 100} \qquad Formula ( F 1 )$$

wherein φₑ is the diameter (in mm) of the wire of the first type at rest, φ_{K} is the diameter (in mm) of the wire of the second type, and Kₘₐₓ is the predetermined maximum elongation rate in percent.

the wire of the first type is twisted upon itself with a specific number of wire turns, wherein the wire turns coil in the opposite of the direction of the turns of the spiral formed by the wire of the first type around the wire of the second type, wherein the number of wire turns per linear metre of the hybrid cable at the maximum elongation rate is between n_{sE} and 3×n_{sE}, preferably between n_{sE} and 2×n_{sE};

each measurement has a precision of less than 0.1%;

for the/each hybrid elastic cable, the reference points of the hybrid elastic cable are fixed relative to one another.

In a second aspect, the invention concerns a method for measuring a variable representative of a circumference or a variation of a circumference of a deformable object, wherein the method comprises the following steps:
arranging at least one hybrid elastic cable that is coiled so as to form at least one turn around the deformable object, wherein the/each hybrid elastic cable comprises a wire of a first type and a wire of a second type,
wherein, for the/each hybrid elastic cable, the wire of the first type has a degree of tenacity lower than that of the wire of the second type, wherein the wire of the second type has a degree of elasticity lower than that of the wire of the first type, wherein the wire of the second type comprises a conductive material,
wherein, for the/each hybrid elastic cable, the wire of the second type is coiled helically around the wire of the first type when the hybrid elastic cable is at rest,
measuring the inductance of the wire of the second type of the cable between two reference points of the hybrid elastic cable,
for the/each hybrid elastic cable, calculating the representative variable at the level of the cable using the inductance of the wire of the second type of the cable being measured.

In particular embodiments, the measurement method comprises one or more of the following characteristics, alone or in any combination technically possible:

the deformable object has a substantially circular cross section at the level of the/each hybrid elastic cable, wherein the representative variable is calculated in the calculating step for the cable using a linear relationship between the inductance measured by the measuring unit and the representative variable.

for the/each hybrid elastic cable, each reference point of the hybrid elastic cable is kept at a fixed position on the deformable object;

the wire of the first type has a resting diameter φₑ, the wire of the second type has a diameter φ_{K}, and the deformable object has a resting circumference Pe at the level of the/each cable (52), such that (φₑ₊φ_{K})<Pe/(10*π).

In a third aspect, the invention also concerns a device for measuring by pressurising the subsoil, comprising:
at least one probe for insertion into a borehole of the subsoil, wherein the probe comprises a deformable cell, and
a transfer unit configured to insert a fluid into the deformable cell,

characterised in that it further comprises an apparatus for measuring a variable representative of a circumference or a variation in a circumference of the deformable cell, wherein the measurement apparatus has the aforementioned characteristics, wherein the/each hybrid elastic cable of the apparatus is coiled and forms at least one turn around the deformable cell of the probe.

In particular embodiments, the measurement device comprises several hybrid elastic cables distributed over the deformable cell at different predetermined longitudinal heights.

In a fourth aspect, the invention also concerns the use of an apparatus to measure the circumference of an inflatable stopper or a packer or to control the dilation of the stopper or packer, wherein the measurement apparatus has the aforementioned characteristics, wherein the/each hybrid elastic cable of the apparatus is coiled and forms at least one turn around the stopper/packer.

In a fifth aspect, the invention also concerns the use of a measuring apparatus as described supra in the field of inductance plethysmography to measure the circumference of a part of the body of an individual or a living being or a variation in the circumference of the part of the body, wherein the measurement apparatus has the aforementioned characteristics, wherein the/each hybrid elastic cable of the apparatus is coiled and forms at least one turn around the part of the body, wherein the part of the body is, e.g., the torso or abdomen.

Other characteristics and advantages of the invention will be seen from the following detailed description, provided by way of example only, which refers to the attached drawings, which show:

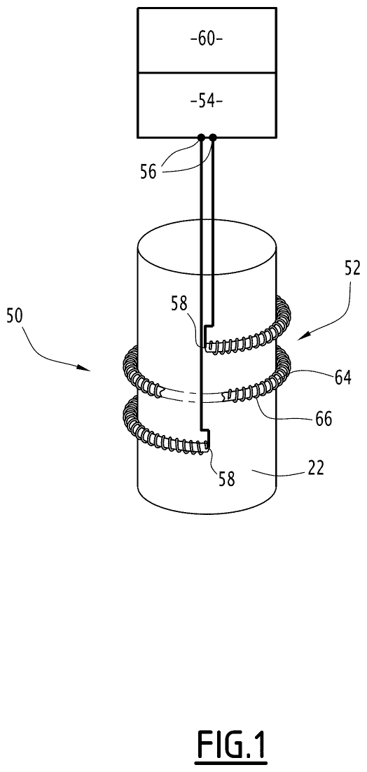

FIG. 1 is a simplified schematic representation of the measurement apparatus according to the invention, mounted around a deformable object;

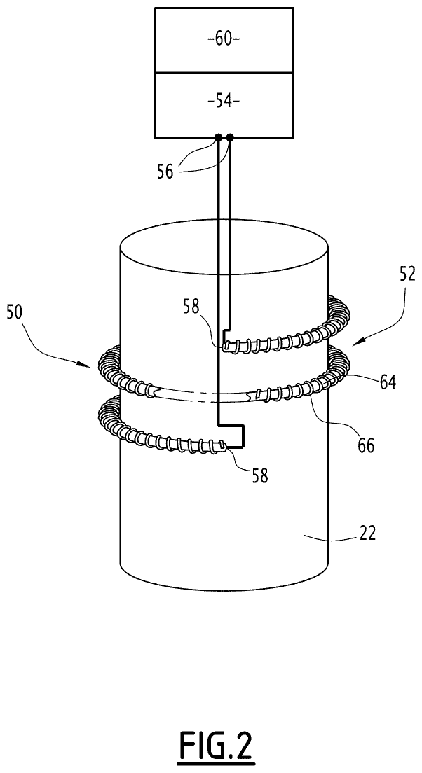

FIG. 2 is a representation similar to that of FIG. 1 following the expansion of the deformable object;

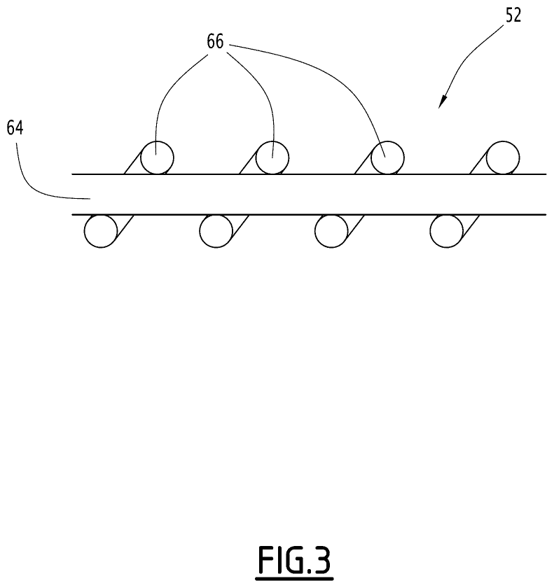

FIG. 3 is a view of a longitudinal section of a hybrid elastic cable of the apparatus of FIG. 1 at rest,

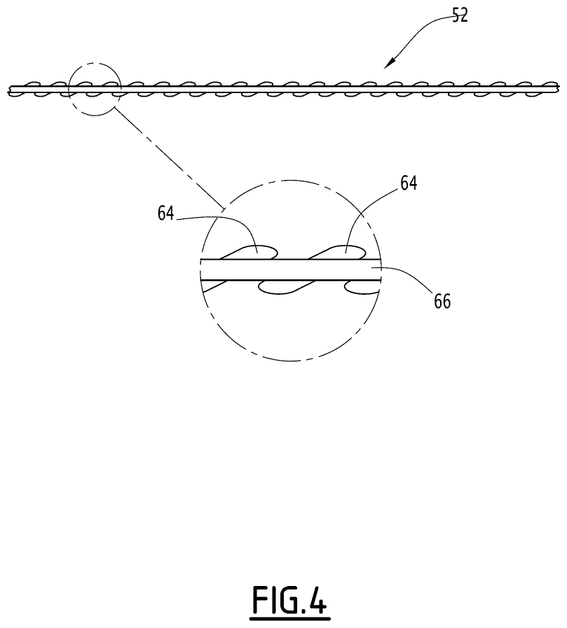

FIG. 4 is a schematic side and longitudinal section view of the hybrid elastic cable of FIG. 3 at a maximum rate of elongation Kₘₐₓ,

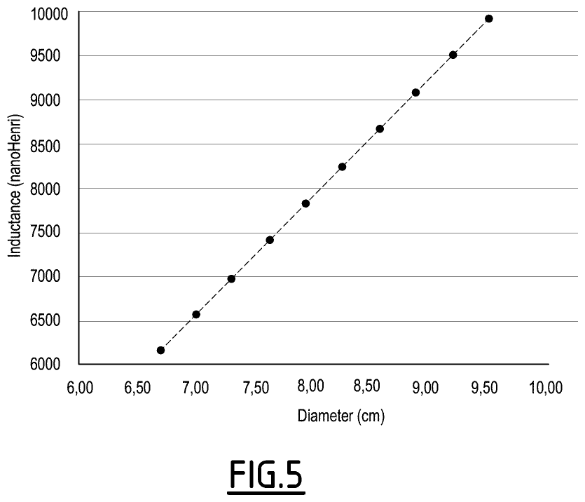

FIG. 5-8 are graphic representations of the inductance of a hybrid elastic cable measured by the measurement unit of FIG. 1 as a function of the diameter of the deformable object of FIG. 1 in different testing conditions;

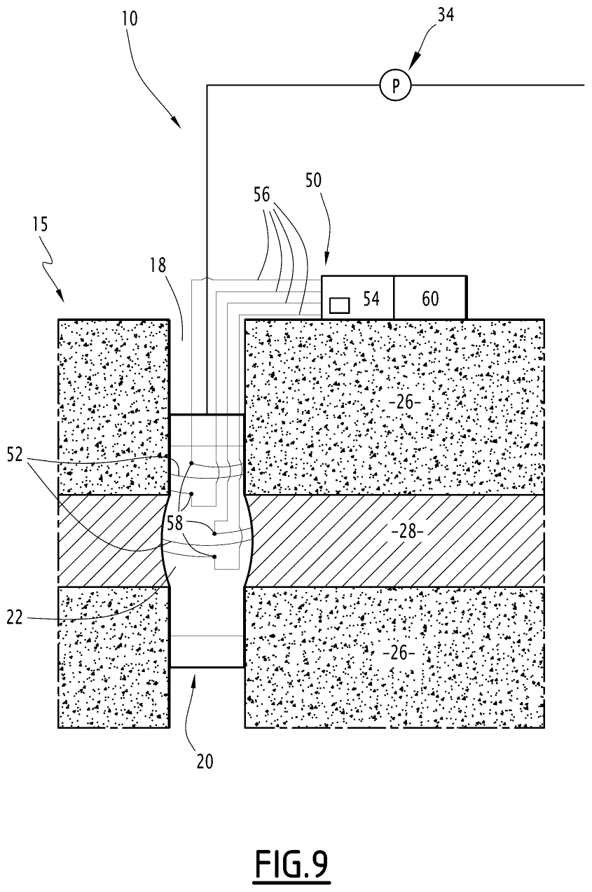

FIG. 9 is a schematic representation of a dilatometric probe equipped with the measurement apparatus of FIG. 1;

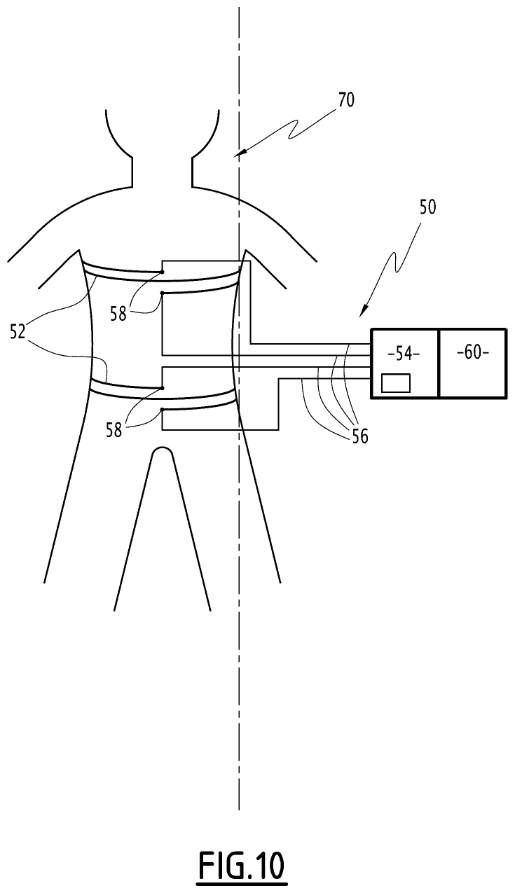

FIG. 10 is a schematic representation illustrating the use of a measurement apparatus according to the invention in the field of inductance plethysmography;

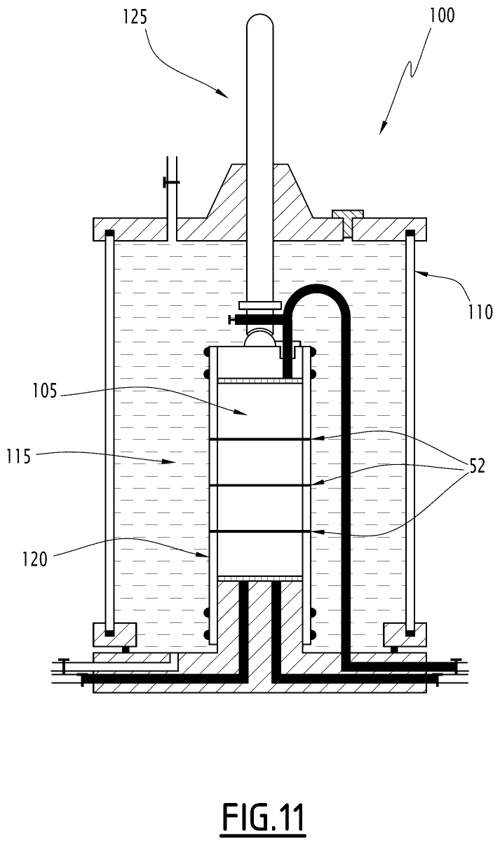

FIG. 11 shows a device for measurement by compressing a soil or rock sample comprising, inter alia, a measurement apparatus according to the invention;

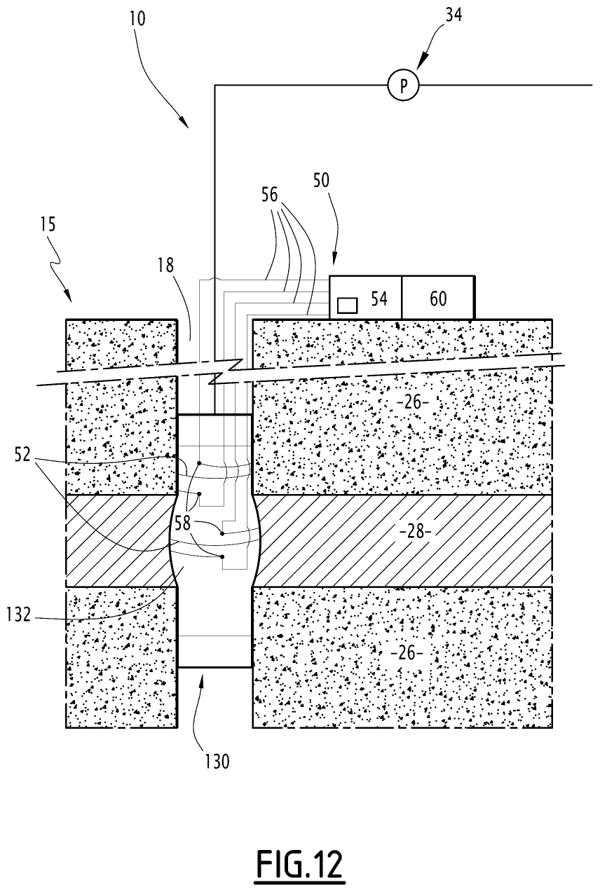

FIG. 12 is a schematic representation illustrating the use of a measurement apparatus according to the invention to measure the circumference of an inflatable stopper or packer; and

FIG. 13 shows the resting and extended states of two known-art inductance plethysmography belts, one with a sinusoidal conductive wire and the other with a zigzagging conductive wire.

The measurement apparatus 50 shown in FIG. 1-4 is intended to measure the circumference or variation in the circumference (expansion) of a deformable object 22.

It is suited for use in a measurement device 10 that pressurizes the subsoil 15, as shown schematically in FIG. 9.

It is also suited for use to measure the circumference of an inflatable stopper or packer or to control the expansion of the stopper/packer, as shown in FIG. 12.

It can also be used in any other field, to measure the circumference or expansion of any other deformable object.

For example, it is well suited for use in the field of inductance plethysmography, as shown in FIG. 10, e.g. to measure the circumference or expansion of a part of the body of an individual or animal. For example, this body part may be the torso, an arm, a thigh, etc.

Likewise, it is suited for use for measurements in simple or triaxial compression tests on a soil or rock sample, as shown in FIG. 11.

‘Deformable object’ refers here to any body, whether inanimate or living, the circumference of which is capable of changing, whether spontaneously or under the effects of an external force.

More specifically, the apparatus 50 is intended to measure a variable representative of the circumference of the deformable object 22. For example, this variable may be the circumference of the deformable object itself. In one variant, it is intended to measure its diameter or radius, in the case of an object having a circular cross-section. In another variant, it measures a variable representative of the variation in the circumference of the deformable object 22, i.e. the expansion of the deformable object 22. In this case, the apparatus measures the variation in the circumference, diameter, or radius of the object.

The apparatus 50 comprises at least one coiled hybrid elastic cable 52 of a predetermined length that forms at least one turn around the deformable object 22 at a predetermined longitudinal height.

The/each hybrid elastic cable 52 makes a certain number of turns around the deformable object 22, typically 1-10, but the number may exceed 10.

The precision of the measurement increases with the number of turns, and increases when the turns are tight, i.e. contiguous.

In FIGS. 1 and 2, the deformable object 22 is cylindrical. In one variant, it has any other shape that allows the hybrid cable to be coiled around the object 22.

The apparatus 50 according to the invention further comprises an inductance measurement unit 54, which is electrically connected via one more pairs of measurement terminals 56 to a pair of reference points 58 of each cable 52, and a calculation unit 60 that is electrically connected to the measurement unit 54.

For example, the reference points 58 of a cable 52 correspond to the ends of the cable 52.

Preferably, the reference points 58 of each cable 52 are in a fixed position on the deformable object 22. The reference points 58 of the hybrid elastic cable are fixed relative to one another. In other words, the distance between the reference points 58 of each cable 52 cannot change substantially as the deformable object 22 deforms.

FIG. 3 is a schematic longitudinal section view of a hybrid elastic cable 52 at rest, as included in the apparatus 50.

The cable 52 comprises a wire of a first type 64 (‘high-elasticity wire’) and a wire of a second type 66 (‘high-tenacity wire’).

The high-elasticity wire 64 has a degree of tenacity lower than that of the high-tenacity wire 66, and the high-tenacity wire 66 has a degree of elasticity lower than that of the high-elasticity wire 64.

In FIG. 1-4, the cross sections of the wires 64 and 66 are not drawn to scale relative to the dimensions of the deformable bodies.

When the hybrid elastic cable is at rest, the wire 66 is coiled helically around the wire 64, as shown in FIG. 3. As the cable 52 extends, the relative positions of the wires 64 and 66 reverse, arriving at a configuration in which, starting at a maximum elongation rate Kₘₐₓ, the wire 64 is wound helically around the stretched wire 66, as shown in FIG. 4.

Thus, when at rest, the high-tenacity wire 66 forms a circular toroidal solenoid having a longitudinal axis. As it extends, the turns of the solenoid move farther and farther apart, and the radius of the turns decreases.

The high-elasticity wire 64 may be selected from wires of the following group: elastomeric wires such as polyurethane wires, elastane wires, or a combination thereof.

The high-tenacity wire 66 comprises a conductive material.

For example, the wire 66 consists of wires enamelled with a conductive material.

In one variant, the wire 66 consists of wires enameled with conductive material lined with a wire selected from the following group: natural fibres, such as cotton, flax, or hemp, glass fibres, carbon yarns, organic fibres such as aramid, para-aramid, polyester, polypropylene, polyamide, aramid, or a combination thereof. The wire selected and the enamelled wires are coiled around one another in a double helix, forming the high-tenacity wire 66.

Preferably, the wire 66 and the wire 64 have elastic moduli in a ratio greater than or equal to 10000.

In a particular exemplary embodiment of the invention, the high-elasticity wire 64 consists of a wire having a longitudinal elastic modulus equal to app. 2 MPa. The high-tenacity wire 66 consists of copper-enamelled wires lined with 1600 dTex aramid wire, marketed under the name Kevlar®, for example, having a longitudinal elastic modulus equal to app. 30000 MPa.

For example, the hybrid elastic cable 52 is of the type described in application WO 2013/110731.

In this example, when fully elongated, the hybrid cable is in a configuration in which the high-elasticity wire is coiled helically around the high-tenacity wire with a number of turns per linear metre of the hybrid cable between n_{sE}−15% and n_{sE}+15%, n_{sE} is determined as a function of the diameter of the high-elasticity wire, the diameter of the high-tenacity wire, and a predetermined maximum elongation rate, based on the following formula:

$${n}_{sE} = \frac{1000}{π ( {φ}_{s} + {φ}_{K} )} \times \frac{\sqrt{{K}_{m ax} \times ( {K}_{m ax} + 200 )}}{{K}_{m ax} + 100} \qquad Formula ( F 1 )$$

wherein φₑ is the diameter (in mm) of the high-elasticity wire at rest, φ_{K} is the diameter (in mm) of the high-tenacity wire, and Kₘₐₓ is the predetermined maximum elongation rate in percent.

Moreover, the high-elasticity wire is twisted onto itself with a specific number of wire turns, wherein the wire turns coil in the opposite of the direction of the turns of the spiral formed by the high-elasticity wire around the high-tenacity wire, which promotes the coiling of the high-tenacity wire around the high-elasticity wire when the hybrid cable returns from its fully extended configuration to its resting configuration.

The number of wire turns per linear metre of the cable at the maximum elongation rate is between n_{sE} and 3×n_{sE}, preferably between n_{sE} and 2×n_{sE}.

The spiral shape of the high-elasticity wire, and its deformation when the hybrid cable relaxes to return to its resting configuration, guide the high-tenacity wire, allowing it to position itself around the high-elasticity wire in an orderly fashion.

Compliance with the above specifications on the number of wire turns of the high-elasticity wire and the number of turns of the high-elasticity wire when the hybrid cable is fully elongated allows for the production of a hybrid cable with favourable behaviour over a range of elongation rates running from 0 to several hundred percent. For example, the predetermined maximum elongation rate of the hybrid cable may be between 100 and 400%. The upper limit may, for example, be defined by the number of contiguous turns of the high-tenacity wire that can be placed over the high-elasticity wire at rest.

Advantageously, the wire of the first type has a resting diameter φₑ, and the wire of the second type has a diameter φ_{K}, such that (φₑ₊φ_{K})<Pe/(10*π), where Pe is the resting circumference of the deformable object at the level of the/each hybrid elastic cable. The inventor believes that such a size ratio contributes to obtaining measurements of increased precision.

In other words, the ratio between the diameter of the hybrid elastic cable and the circumference of the deformable object is less than 10*π.

‘Resting circumference’ refers to the minimum circumference of the deformable object within the measurement range in question.

In one variant, the cable 52 is not of the type described in application WO 2013/110731, and is instead of any other suitable type.

The measurement unit 54 is suited to measure the inductance of the wire 66 of the hybrid elastic cable 52 between the two reference points 58 of the cable 52 for each cable 52.

For example, the measurement unit 54 is, for example [sic], an inductance meter known to persons skilled in the art.

The inductance meter may, for example, be a commercial multimeter of the type Keysight U1733C or Siborg LCR Reader, or any other equivalent model.

The measurement unit 54 is also suited to transmit each inductance measured to the calculating unit 60.

For each hybrid elastic cable 52, the calculating unit 60 is configured to calculate the variable representative of the circumference or variation in circumference of the deformable object 22 at the predefined level on the cable 52 using the inductance of the high-tenacity wire 66 of the hybrid elastic cable being measured.

The tests carried out have shown that the inductance of the high-tenacity wire 66 of the cable 52, and, more specifically, the inductance measured by the measurement unit 54 between the reference points 58 of the hybrid elastic cable 52, is proportional to the circumference of the deformable object 22 at the longitudinal height of a cable 52. More precisely, it varies in a linear fashion with the circumference of the deformable object 22 at the longitudinal height of a cable 52. This is shown in FIG. 5-8.

The curve in FIG. 5 was obtained for measurements with a measurement apparatus 50 comprising a single hybrid elastic cable 52 that was coiled and formed three turns around a deformable cylindrical cell 22. The inductance measurements were carried out for a diameter of the deformable cell 22 that varied between 65 and 95 mm.

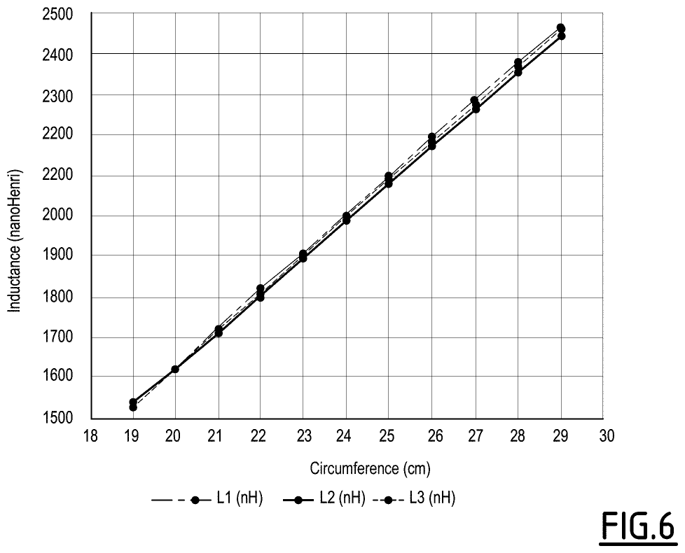

The curves in FIG. 6 were obtained for measurements with a measurement apparatus 50 comprising three hybrid elastic cables 52 (curves L1, L2, and L3), each of which was coiled and formed three turns around a deformable cylindrical cell 22. The turns are contiguous. The hybrid cables were arranged axially at three different levels on the cell.

The resting diameter φₑ of the wire of the first type (high elasticity) is 0.63 mm. The diameter φ_{K} of the wire of the second type (high tenacity) is 0.4 mm. This wire is a multistrand copper wire consisting of 9 strands. The predetermined maximum elongation rate Kₘₐₓ is 170%. The number n_{sE} of turns of the wire of the second type at rest around the wire of the first type per linear metre is 750 turns/m.

The inductance measurements were carried out for a circumference of the deformable cell 22 that varied between 19 and 29 cm, i.e. an expansion rate of 150%. The test was carried out by dilating, and then retracting, the deformable cell.

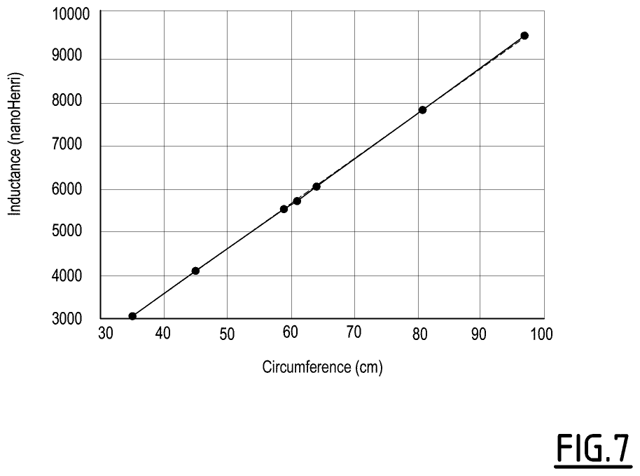

The curve in FIG. 7 was obtained for measurements with a measurement apparatus 50 comprising a single hybrid elastic cable 52 that was coiled and formed three turns around a deformable cylindrical cell 22. The turns are contiguous.

The resting diameter φₑ of the wire of the first type (high elasticity) is 0.63 mm. The diameter φ_{K} of the wire of the second type (high tenacity) is 0.4 mm. This wire is a multistrand copper wire consisting of 9 strands. The predetermined maximum elongation rate Kₘₐₓ is 300%. The number n_{sE} of turns of the wire of the second type at rest around the wire of the first type per linear metre is 1200 turns/m.

The inductance measurements were carried out for a circumference of the deformable cell 22 that varied between 35 and 98 cm, i.e. an expansion rate of 277%.

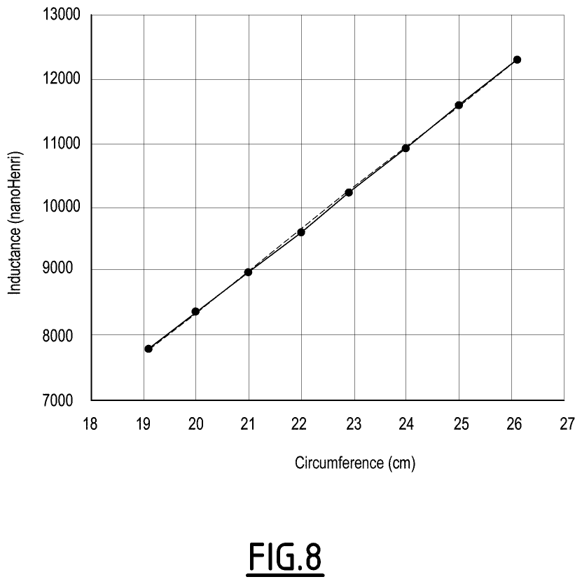

The curve in FIG. 8 was obtained for measurements with a measurement apparatus 50 comprising a single hybrid elastic cable 52 that was coiled and formed 9 turns around a deformable cylindrical cell 22. The inductance measurements were carried out for a circumference of the deformable cell 22 that varied between 19 and app. 26 cm, i.e. an expansion rate of 137%. The turns are contiguous.

The resting diameter φₑ of the wire of the first type (high elasticity) is 0.63 mm. The diameter φ_{K} of the wire of the second type (high tenacity) is 0.4 mm. This wire is a multistrand copper wire consisting of 9 strands. The predetermined maximum elongation rate Kₘₐₓ is 170%. The number n_{sE} of turns of the wire of the second type at rest around the wire of the first type per linear metre is 750 turns/m.

In all cases, a linear relationship was observed between the inductance measured and the circumference of the deformable cell. The determination coefficient R² is in all cases greater than 99.98%.

Likewise, the inductance measured by the measurement unit 54 between the reference points 58 of the cable 52 varies in a linear fashion with the diameter of the deformable object 22 when it has a substantially circular cross section at the predetermined height.

It is proportional to the inductance of the wire 66 of the cable 52, and, more specifically, to the inductance measured by the measurement unit 54 between the reference points 58 of the hybrid elastic cable 52.

As such, the calculating unit 60 is then configured to calculate the representative variable for each hybrid elastic cable 52 using a linear relationship between the inductance measured by the measuring unit 54 and the representative variable. This linear relationship corresponds to a predetermined characteristic line of the cable 52. The characteristic line corresponds to the line representing the inductance measured as a function of the circumference.

This characteristic line is determined during a prior calibration step using tubes with calibrated cross-sections. This contributes to very good measurement precision.

Advantageously, when the deformable object 22 is substantially circular at the predetermined height, for each hybrid elastic cable 52, the calculating unit 60 is configured to calculate a diameter of the deformable object 22 as a function of the inductance of the high-tenacity wire 66 of the hybrid elastic cable 52 being measured, e.g. via a predefined characteristic line that represents the inductance measured as a function of diameter.

In FIG. 1, the deformable object 22 has a first circumference. The hybrid elastic cable 52 has a first elongation. The high-tenacity wire 66 is solenoid-shaped with a closed contour and a circumference equal to that of the deformable object. It forms turns spaced apart by a first distance.

In FIG. 2, the deformable object 22 has undergone expansion. The hybrid elastic cable 52 has a second elongation, greater than the first. The high-tenacity wire 66 remains solenoid-shaped with a closed contour and a circumference equal to that of the deformable object. The turns of the wire are spaced apart by a second distance, greater than the first.

Thus, the elongation of a cable 52 of the measurement apparatus 50 varies as a function of the circumference of the deformable object 22 at the predetermined longitudinal height on the cable 52. The shape of the wire 66 also varies as a function of the circumference of the deformable cell 22. The diameter of the solenoid formed by the wire 66 increases with the circumference of the deformable object 22. This is possible because the turns move away from one another. The inductance of the wire 66 is essentially a function of the circumference of the solenoid, which follows that of the deformable cell 22.

Because the hybrid elastic cable is capable of substantial elongation, it is possible to carry out measurements over a wide range of expansion values going up to at least a factor of 1:3 of the circumference without losing measurement precision. The measurement sensors currently marketed for inductance plethysmography (commonly known as plethysmography belts) only operate within a much smaller range of expansion values, typically from 15-40%, and repeated cyclical stresses at greater elongation rates may degrade the sensors and thus the quality of the measurements.

Indeed, the hybrid cable used in this invention behaves like an elastic wire with constant elasticity up to a predetermined maximum elongation, and beyond that, when the predetermined elongation has been reached, it behaves like a high tenacity wire, with very low elongation and great breaking strength.

The hybrid cable used in this invention, which is coiled and forms at least one turn around the deformable object, is thus designed and selected so that it only operates within its elastic range, thus offering very low resistance over the entire intended measurement range and avoiding resistance to the expansion of the deformable object. To this end, it is sufficient for the hybrid cable to be designed such that its maximum elongation capacity is greater than the maximum expansion factor of the deformable object being measured; this is possible for expansion factors of the deformable object of up to 1:3 and beyond.

In one of the experimental operations on an inflatable body with a substantially circular resting cross-section and a diameter of 200 mm, it was found that the uncertainty in circumference measurements arises primarily from that of the inductance measurement 54. Thus, when the measurement unit 54 was suited to provide inductance measurements with a precision of less than 10 nH, we were able to ensure that each diameter measurement carried out by the measurement apparatus 50 had a precision of less than 100 μm.

These measurements are reproducible without any loss of precision.

In the curve of FIG. 5, a measurement error of 10 nH corresponds to a 225 μm measurement error on the circumference, i.e. a 75 μm diameter measurement error.

Without being bound by this theory, the inventor believes that the following elements contribute to this excellent precision:
the fact that the hybrid cable can undergo numerous elongation/retraction cycles without any deterioration in the arrangement of the high-tenacity wire, i.e. the conductive wire;
the fact that the hybrid cable operates in its range of very low elongation resistance, where the conductive wire is not subject to significant mechanical stresses;
the fact that the inductance of the hybrid cable is strictly a function of the circumference of the torus formed by the conductive wire, which very precisely follows the circumference of the deformable object;
the fact that the diameter of the conductive wire (our the sum of the diameters of the wire of the first type and the diameter of the wire of the second type), is selected to be much lower than the diameter of the deformable object;
the fact that the reference points are fixed relative to one another in order to avoid any variable parasitic inductance attributable to the loop formed by the terminals of the measurement unit itself (inductance meter);
the fact that the relationship between inductance and circumference remains linear regardless of the number of turns of the hybrid cable, for a low number of turns of the hybrid cable around the deformable object, with the maximum number of turns being selected such that the longitudinal distance (along the deformable object) between the first and last turn of the coiled hybrid cable remains less than the resting diameter of the deformable object;
the fact that the inductance value measured at the reference points is independent of the number of turns (of the wire of the second type around the wire of the first type) per linear metre of the hybrid cable.

The apparatus 50 is particularly simple to operate on deformable objects 22 of all sorts.

Although this is not indispensable, the hybrid cables may also be mounted on an elastic sheath that is then threaded around the deformable object. In this case, the hybrid cables and the elastic sheath need not be glued or sewn together.

Several applications will now be described by reference to FIG. 9-12.

The measurement device 10 shown in FIG. 9 is a dilatometric measurement device or a pressiometric measurement device intended, respectively, for dilatometric or pressiometric testing.

For example, the device 10 is a Ménard pressiometric measurement device conforming to standard NFP 94-110-1.

The device 10 comprises a probe 20 intended for insertion into a longitudinal borehole 18 in the subsoil 15. In the example of FIG. 9, the probe 20 comprises a cell 22 in the shape of a tube with a central longitudinal axis that can be deformed by injecting fluid. The deformable cell constitutes a deformable object within the meaning of this invention.

The probe may, for example, be a pressiometric probe.

For this application, the hybrid cables may advantageously be covered with a protective sheath, e.g. one made of an elastomer, in order to avoid direct contact with the ground.

By way of example, the subsoil 15 comprises a layer 28 of a first type of material between two layers 26 of a second type of material.

For example, the probe 20 of the device 10 according to the invention is a Ménard probe conforming to standard NFP 91-110-1, advantageously an FC 60 Francis COUR with a flexible sheath.

The FC 60 Francis COUR probe is described, e.g., in the Applicant's patents FR 3 009 841 and FR 2 910 047.

The device 10 also comprises a transfer unit 34 such as a pump, configured to insert a fluid into the deformable cell 22.

Typically, the fluid is an incompressible fluid, such as water. In one variant, the fluid is a gas.

The device 10 according to the invention further comprises the measurement apparatus 50. It is configured to measure a variable representative of the circumference or a variation in the circumference of the deformable object 22, here the deformable cell, at at least one predetermined longitudinal height on the deformable object 22.

Advantageously, the apparatus 50 is configured to measure the representative variable at various predetermined longitudinal heights on the deformable object 22, here the deformable cell.

In this case, for the/each predetermined longitudinal height, the apparatus 50 comprises one hybrid elastic cable 52 that is coiled and forms at least one turn around the deformable object 22 at the predetermined longitudinal height.

By way of example, the apparatus 50 of FIG. 9 comprises two cables 52.

In other embodiments, the apparatus 50 comprises 3 or 4 cables 52, or more than 4 cables 52.

The pairs of reference points 58 of each cable 52 are electrically connected to the inductance measurement unit 54.

Each hybrid elastic cable 52 makes a certain number of turns around the deformable object 22, typically 1-10, but the number may exceed 10.

The conductors used to form the wires of the second type may also advantageously be selected such that they are flexible, e.g. enamelled multistrand conductors.

This sheath is a sleeve threaded around the deformable cell 22, and covering at least the full length of the cell on which the hybrid cable is plated.

Each hybrid cable is connected to an electronic housing near the probe, which measures the inductance of each conductive wire simultaneously, or, advantageously, one by one, and transmits the measured inductance values to the surface (via a wireless connection, an electrical cable, or a fibre optic). The measurements are displayed and/or recorded on the surface.

The probe may be protected by a flexible sheath, e.g., made of an elastomer (not shown), to avoid direct contact between the hybrid cables and the ground, and thus avoid damaging them.

The probe is also equipped with a pressure sensor (not shown), which directly measures the pressure in the deformable cell or the conduit that allows the deformable cell to be supplied from the surface. A recording and display system then allows the curve connecting the pressure measured and the diameter of the probe at each level to be traced.

The operation of a device 10 will now be described by reference to the example of a subsoil pressurisation test.

First, the probe 20 is inserted into a borehole 18 in the subsoil 15 such that the probe 20 is positioned at a defined depth (measurement station).

The pressiometric/dilatometric tests are typically carried out at different depths along the borehole 18.

The deformable cell 22 is pressurised at increasing pressure levels by inserting fluid via the transfer unit 34.

The pressurisation is carried out, e.g., at successively increasing levels.

The pressure inside the deformable cell 22 may vary, e.g., from 1-500 bar.

The diameter of the deformable cell 22 varies as a function of the pressure level and the type of subsoil 15 material.

The elongation of each cable 52 of the measurement apparatus 50 varies as a function of the circumference of the deformable cell 22 at the predetermined longitudinal height on the cable 52. The inductance of the high-tenacity wire 66 varies as a function of the circumference of the deformable cell 22.

Then, the measurement unit 54 measures the inductance of the wires 66 of each cable 52, and transmits it to the calculating unit 60; then, the calculating unit 60 calculates the representative variable for the respective cable 52 as a function of the inductance of the wire 66 measured, e.g. by means of a characteristic line of the cable 52.

The relationship between the inductance of each hybrid cable and the representative variable sought, which was previously established by calibration for each of the hybrid cables, allows for real-time display (and, if necessary, recording) of the development of the diameter of the deformable cell at each of its levels as it expands and contracts.

The steps described above are successively repeated for each pressure level.

In the case of a pressiometric test, a pressiometric curve or expansion curve is then obtained by representing the pressure measured in the deformable cell 22 on the abscissa axis and the variation in diameter over the course of the pressiometric test on the ordinate axis.

This curve is then analysed in order to determine the mechanical properties of the subsoil 15.

Typically, the probe 20 is then vertically displaced within the borehole 18 to be positioned at the level of the next measurement station, and the steps of the method are repeated.

Advantageously, the invention comprises a step of calibrating the calculating unit 60 prior to the insertion of the probe 20 into the borehole 18. During this step, the transfer unit 36 inserts fluid from the fluid reservoir 30 into the deformable cell 22 until the cell 22 reaches a predetermined diameter. The measurement unit 54 then measures the corresponding inductance for each hybrid elastic cable 52.

This operation is carried out for different diameters. For each hybrid elastic cable 52, a curve of a characteristic of the cable 52 is obtained by indicating the representative variable, e.g. the diameter of the deformable cell 22, on the abscissa axis, and the inductance measured on the ordinate axis. Such a curve is shown in FIG. 5. For example, the calculating unit 60 may carry out a linear regression for each cable 52 in order to obtain the characteristic line of the cable 52 allowing for the calculation of the corresponding representative variable as a function of an inductance measured.

The value of measuring a representative variable such as the diameter of the probe at different levels during its inflation is to detect non-homogeneous inflation, which would result from heterogeneous soil, with layers offering different levels of resistance at different levels.

The probes currently on the market do not allow for such measurements at different levels, and are thus liable to deteriorate upon inflation within highly heterogeneous soil or to give inaccurate soil resistance measurements.

By way of example, the probe 20 in FIG. 1 is positioned at a depth where one of the layers 26 and the layer 28 are superimposed. The deformable cell 22 of the probe 20 then includes a part that is in contact with one of the layers 26 and another part that is in contact with the layer 28. A first of the two hybrid electric cables 52 of the apparatus 50 is coiled around the deformable cell 22 at the level of the part that is in contact with the layer 26, and a second of the two cables 52 is coiled around the cell 22 at the level of the part that is in contact with the layer 28.

In the example of FIG. 1, the part of the deformable cell 22 that is in contact with the layer 26 has a smaller diameter than the part of the cell 22 that is in contact with the layer 28. The inductance measured for the first of the two cables 52 will thus be smaller than the inductance measured for the second of the two cables 52, which translates in to different physical properties for the layers 26 and 28.

Thus, it will be understood that, by increasing the number of cables 52 of the apparatus 50 that are distributed over the deformable cell 22, it is possible to obtain better information on the physical properties of the subsoil 15 that is in contact with the cell 22, in particular when the subsoil 15 comprises several layers of different types of material.

Thus, the apparatus 50 according to the invention allows for the measurement of a representative variable such as the circumference or diameter of the deformable cell 22 at different longitudinal heights in a precise and simple manner, with this representative variable being variable as a function of the quantity of fluid inserted into the cell 22.

Lastly, the apparatus 50 according to the invention allows for measurements of the representative variable of the deformable cell 22 without the need for adjustments or repositioning between different measurements.

A second example of the application of the apparatus 50 will now be described by reference to FIG. 10.

In this example, the apparatus 50 is used in the field of inductance plethysmography to measure the circumference of a part of the body of an individual 70 or any other living being. For example, the apparatus 50 is used to measure a circumference around the torso of an individual 70, as shown in FIG. 10.

In this use case, the apparatus 50 is as described supra. The deformable object 22 corresponds to the body part of the individual 70. The apparatus 50 then comprises at least one hybrid elastic cable 50 that is coiled around the individual's 70 torso and forms at least one turn around the individual's 70 torso at at least one predetermined height.

By way of example, the apparatus 50 of FIG. 10 comprises two cables 52. For example, one of the cables 52 is placed at the level of the chest cavity of the individual 70, and the other is placed at the level of their abdomen.

When the apparatus 50 is used in this manner, the measurement unit 54 measures the inductance of the high-tenacity wires 66 of each cable 52 and transmits it to the calculating unit 60; then, the calculating unit 60 calculates the circumference of the torso of the individual 70 at the level of each cable 52 using the corresponding inductance reading.

In a variant not shown, the apparatus 50 is used to measure a circumference of an arm or a leg.

The apparatus 50 then allows for precise monitoring of the circumference of a part of an individual's body part over time without having to adjust or reposition it between measurements.

The apparatus 50 then allows for precise measurements of the circumference of a part of the body of an individual over substantial circumference ranges and for substantial diameters.

A third example of the application of the apparatus 50 will now be described by reference to FIG. 11.

In this example, the apparatus 50 is used for measurements in simple or triaxial compression tests on a soil or rock sample 105 in order to measure a circumference of the soil/rock sample 105.

FIG. 11 shows a triaxial compression device 100 for measuring the sample 105.

The device 100 comprises a chamber 110 suited to contain a fluid 115, with the sample 105 positioned within the chamber 110.

Advantageously, the device 100 comprises a membrane 120 positioned within the chamber 110 and containing the sample 105.

The device 100 further comprises a plunger 125 configured to exert an axial force on the sample 105 at a specified pressure.

The device 100 further comprises a measurement apparatus 50 configured to measure the circumference of the sample 105.

In this use case, the apparatus 50 is as described supra. The deformable object 22 corresponds to the sample 105. The apparatus 50 then comprises at least one hybrid elastic cable 50 that is coiled around the sample 105 and forms at least one turn around the sample 105 at at least one predetermined height. By way of example, it comprises three hybrid elastic cables 52.

When the device 100 comprises a membrane 120, the at least one cable 52 is coiled around the membrane 120.

For clarity's sake, FIG. 11 only shows the cables 52 of the apparatus 50.

Advantageously, the sample 105 is cylindrical, and the at least one hybrid elastic cable 52 is coiled around the axis of the sample 105.

The chamber 110 may, for example, be cylindrical, with the axis of the sample 105 then being aligned with the axis of the chamber 110 and the plunger 125 being configured to exert the axial force along the axis of the sample 105.

The circumference measurements around the sample 105 are carried out for various specified pressure levels exerted by the plunger 125 on the sample 105 in order to obtain a pressiometric curve of the compression of the sample 105 as a function of the specified pressure exerted on the sample 105. This curve is then analysed in order to determine the mechanical properties of the sample 105.

A fourth example of the application of the apparatus 50 will now be described by reference to FIG. 12.

In this example, the apparatus 50 is used to measure the perimeter of an inflatable stopper or a packer 130 or to control the dilation of the stopper or packer 130.

The inflatable stopper 130 has the same general structure as the pressiometric probe 20 described supra. It is typically used in the oil & gas, geothermal, or geotechnological field to seal a well or borehole 28.

The inflatable stopper 130 comprises a cell 132 that is deformable by injecting fluid in the form of a tube with a central longitudinal axis. The deformable cell constitutes a deformable object within the meaning of this invention.

The apparatus 50 is of the type described supra. It comprises at least one hybrid elastic cable 52 coiled around the inflatable stopper 130. Typically, it comprises a plurality of cables 52 that are coiled around the deformable cell 132 and form at least one turn around the deformable cell 132 at different predetermined longitudinal heights.

Controlling the expansion of the inflatable stopper/packer serves in particular to avoid bursting due to excessive expansion of the stopper/packer.

The invention also concerns a method for measuring a variable representative of a perimeter or a variation of a perimeter of a deformable object 22, wherein the method comprises the following steps:
arranging at least one hybrid elastic cable 52 that is coiled so as to form at least one turn around the deformable object 22, wherein the/each hybrid elastic cable 52 comprises a wire 64 of a first type and a wire 66 of a second type,
wherein, for the/each hybrid elastic cable 52, the wire 64 of the first type has a degree of tenacity lower than that of the wire 66 of the second type, wherein the wire 66 of the second type has a degree of elasticity lower than that of the wire 64 of the first type,
wherein the wire 66 of the second type comprises a conductive material,
wherein, for the/each hybrid elastic cable 52, the wire 66 of the second type is coiled helically around the wire 64 of the first type when the hybrid elastic cable 52 is at rest,
measuring the inductance of the wire 66 of the second type of the cable 52 between two reference points 58 of the cable 52,
for the/each cable 52, calculating the representative variable at the level of the cable 52 using the inductance of the wire 66 of the second type of the cable 52 being measured.

The method is particularly suited for use with the measurement apparatus described supra. Conversely, the measurement apparatus is specifically designed to carry out the method.

The method is applicable, in particular, to the four use cases described supra: measurements by pressurising the subsoil, measuring the circumference of an inflatable stopper or packer, inductance plethysmography, simple or triaxial compression tests on a soil or rock sample.

The representative variable measured is as described supra.

The hybrid elastic cable is as described supra.

In particular, the thread of the first type has a resting diameter φₑ, and the thread of the second type has a diameter φ_{K}, such that (φₑ₊φ_{K})<Pe/(10*π), where Pe is the resting circumference of the deformable object at the level of the/each hybrid elastic cable 52.

The inductance measurement is carried out as described supra.

The deformable object is as described supra.

In particular, when the deformable object 22 comprises a cross-section that is substantially circular at the height of the/each hybrid elastic cable 52, the representative variable is calculated in the calculating step for the cable 52 using a linear relationship between the inductance measured by the measuring unit 54 and the representative variable.

This linear relationship is as described supra.

Advantageously, for the/each hybrid elastic cable 52, each reference point 58 of the hybrid elastic cable is kept at a fixed position on the deformable object 22.

1. An apparatus for measuring a variable representative of a circumference or a variation of a circumference of a deformable object, wherein the apparatus comprises an inductance measurement unit, and
at least one hybrid elastic cable coiled and arranged to form at least one winding around the deformable object, wherein the/each hybrid elastic cable comprises a wire of a first type and a wire of a second type,
wherein, for the/each hybrid elastic cable, the wire of the first type has a degree of tenacity lower than that of the wire of the second type, wherein the wire of the second type has a degree of elasticity lower than that of the wire of the first type, wherein the wire of the second type comprises a conductive material,
wherein, for the/each hybrid elastic cable, the wire of the second type is coiled helically around the wire of the first type when the hybrid elastic cable is at rest,
wherein, for the/each hybrid elastic cable, the measurement unit comprises a pair of measuring terminals electrically connected to two reference points of the cable and is suited to measure the inductance of the wire of the second type of the cable between the two reference points of the cable,
wherein the apparatus further comprises a calculator electrically connected to the measurement unit and configured to calculate the representative variable for the/each cable around the cable using the inductance of the wire of the second type of the cable being measured.

2. The apparatus according to claim 2, wherein the deformable object comprises a substantially circular cross-section at the level of the/each cable, and the calculator is configured to calculate the representative variable for a cable using a linear relationship between the inductance measured by the measuring unit and the representative variable.

3. The apparatus according to claim 1, wherein, when fully elongated, the hybrid cable is in a configuration in which the wire of the first type is helically coiled around the wire of the second type with a number of turns per linear meter of the cable between n_{sE}−15% and n_{sE}+15%, wherein n_{sE} is determined as a function of the diameter of the wire of the first type, the diameter of the wire of the second type, and a predetermined maximum elongation rate, based on the following formula:
$${n}_{sE} = \frac{1000}{π ( {φ}_{s} + {φ}_{K} )} \times \frac{\sqrt{{K}_{m ax} \times ( {K}_{m ax} + 200 )}}{{K}_{m ax} + 100} \qquad Formula ( F 1 )$$
wherein φₑ is the diameter (in mm) of the wire of the first type at rest, φ_{K} is the diameter (in mm) of the wire of the second type, and Kₘₐₓ is the predetermined maximum elongation rate in percent.

4. The apparatus according to claim 3, wherein the wire of the first type is twisted upon itself with a specific number of wire turns, wherein the wire turns coil in the opposite of the direction of the turns of the spiral formed by the wire of the first type around the wire of the second type, wherein the number of wire turns per linear meter of the hybrid cable at the maximum elongation rate is between n_{sE} and 3×n_{sE}.

5. The apparatus according to claim 1, wherein each measurement has a precision of less than 0.1%.

6. The apparatus according to claim 1, wherein, for the/each hybrid elastic cable, the reference points of the cable are fixed relative to one another.

7. A method for measuring a variable representative of a circumference or a variation of a circumference of a deformable object, wherein the method comprises the following steps:
arranging at least one hybrid elastic cable that is coiled so as to form at least one turn around the deformable object, wherein the/each hybrid elastic cable comprises a wire of a first type and a wire of a second type,
wherein, for the/each hybrid elastic cable the wire of the first type has a degree of tenacity lower than that of the wire of the second type, wherein the wire of the second type has a degree of elasticity lower than that of the wire of the first type, wherein the wire of the second type comprises a conductive material,
wherein, for the/each hybrid elastic cable, the wire of the second type is coiled helically around the wire of the first type when the hybrid elastic cable is at rest,
measuring the inductance of the wire of the second type of the cable between two reference points of the cable,
for the/each cable, calculating the representative variable at the level of the cable using the inductance of the wire of the second type of the cable being measured.

8. The method according to claim 7, wherein the deformable object comprises a substantially circular cross-section at the level of the/each cable, wherein the representative variable is calculated in the calculating step for the cable using a linear relationship between the inductance measured by the measuring unit and the representative variable.

9. The method according to either of claim 7, wherein, for the/each hybrid elastic cable, each reference point of the hybrid elastic cable are kept in a fixed position on the deformable object.

10. The method according to the claim 7, wherein the wire of the first type has a resting diameter φₑ, and the wire of the second type has a diameter φ_{K}, wherein the deformable object has a resting circumference Pe at the level of the/each cable, such that (φₑ₊φ_{K})<Pe/(10*π).

11. A device for measuring by pressurizing the subsoil, comprising:
at least one probe for insertion into a borehole of the subsoil, wherein the probe comprises a deformable cell, and
a transfer unit configured to insert a fluid into the deformable cell, and
an apparatus according to claim 1, wherein the/each hybrid elastic cable of the apparatus is coiled around the deformable cell of the pressiometric probe and forms at least one turn around the deformable cell of the pressiometric probe.

12. A measuring method in the field of inductance plethysmography to measure the circumference of a part of the body of an individual or a living being or a variation in the circumference of the part of the body, the method comprising:
providing the apparatus according to claim 1,
coiling the/each hybrid elastic cable around the part of the body, and
forming at least one turn around the part of the body, wherein the part of the body is the torso or abdomen.

13. A method to measure the circumference of an inflatable stopper or a packer or to control the dilation of the stopper or packer, the method comprising:
providing the apparatus according to claim 1,
coiling the/each hybrid elastic cable around said stopper or packer, and
forming at least one turn around said stopper or packer.

14. The measuring method according to claim 12, wherein the part of the body is torso or abdomen.

15. The apparatus according to claim 4, wherein the number of wire turns per linear meter of the hybrid cable at the maximum elongation rate is between n_{sE} and 2×n_{sE}.
